(19) United States
(12) Patent Application Publication
(10) Pub. No.: US 2009/0259130 A1
(43) Pub. Date: Oct. 15, 2009

(54) OPTICAL TOMOGRAPHY MEASUREMENT USING AN ADAPTED BRIM FOR THE RECEIVING VOLUME

(75) Inventors: Martinus Bernardus Van Der Mark, Eindhoven (NL); Willem Peter Van Der Brug, Eindhoven (NL); Tim Nielsen, Hamburg (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/439,726

(22) PCT Filed: Sep. 5, 2007
(86) PCT No.: PCT/IB07/53568

§ 371 Date: Mar. 3, 2009

(30) Foreign Application Priority Data
Sep. 7, 2006 (EP) 06120317.0

(51) Int. Cl. A61B 6/00

(52) U.S. Cl. 600/476

(57) ABSTRACT

The invention relates to a device for imaging an interior of a turbid medium comprising: a) a receiving volume for receiving at least a part of the turbid medium, with the receiving volume being bound by a boundary having an opening bound by a brim, b) a light source for irradiating the turbid medium, and c) a photodetector unit for detecting light emanating from the volume as a result of irradiating the turbid medium. The device is adapted such that the brim is arranged for optically coupling the light source to the turbid medium and the turbid medium to the photodetector unit. One embodiment of the invention relates to a device for imaging an interior of a female breast comprising a cup-like receptacle (20) for accommodating the breast. The receptacle (20) comprises a convex shaped brim (60) arranged for optically coupling the light source to the patient's (55) breast and the breast to the photodetector unit.

### FIELD OF THE INVENTION

The invention relates to a device for imaging an interior of a turbid medium comprising:

a) a receiving volume for receiving at least a part of the turbid medium, said receiving volume comprising an opening bound by a brim;
b) a light source for irradiating the turbid medium;
c) a photodetector unit for detecting light emanating from the receiving volume as a result of irradiating the turbid medium.

The invention also relates to a second receptacle, arranged to be inserted into a first receptacle with the first receptacle being comprised in a device for imaging an interior of a turbid medium, said device comprising a light source for irradiating the turbid medium and a photodetector unit for detecting light emanating from the turbid medium as a result of irradiating the turbid medium and with the second receptacle bounding a receiving volume for receiving at least a part of the turbid medium, said receiving volume having an opening bound by a brim with the brim being arranged for optically coupling the light source to the turbid medium and the turbid medium to the photodetector unit.

The invention also relates to a medical image acquisition device comprising:

a) a receiving volume for receiving at least a part of a turbid medium, said receiving volume comprising an opening bound by a brim;
b) a light source for irradiating the turbid medium;
c) a photodetector unit for detecting light emanating from the receiving volume as a result of irradiating the turbid medium.

### BACKGROUND OF THE INVENTION

An embodiment of a device of this kind is known from U.S. Pat. No. 6,327,488 B1. The known device can be used for imaging an interior of a turbid medium, such as biological tissues. In medical diagnostics the device may be used for imaging an interior of a female breast. The receiving volume receives a turbid medium, such as a breast. The turbid medium is then irradiated with light from the light source, resulting in light traveling through the turbid medium. Typically, light having a wavelength within the range of 400 nm to 4000 nm is used for irradiating the turbid medium. The measurement principle is that transilluminating the turbid medium from one side to the opposite side yields information concerning an interior of the turbid medium. Light emanating from the receiving volume as a result of irradiating the turbid medium is detected by the photodetector unit and used to derive an image of an interior of the turbid medium.

### SUMMARY OF THE INVENTION

It has been found that the known device does not always provide sufficient information about the whole interior of the turbid medium relevant during a measurement. It is an object of the invention to provide a device that has the possibility to provide more information than the known device about the whole interior of the turbid medium relevant during a measurement.

According to the invention this object is realized in that at least a part of the brim is arranged for optically coupling the light source to the turbid medium and the turbid medium to the photodetector unit. With the known device it is very difficult to image an interior of the turbid medium on the brim. In medical diagnostics, where the device may be used for imaging an interior of a female breast, breast cancer is often found on the lateral side of the breast close to a patient's armpit. This is where the lymph nodes are. In the known device no measurements are performed on the brim. This implies that currently a part of the breast, or breast related tissue, is not imaged during an examination. The device according to the invention enables imaging of an interior of the turbid medium near or on the brim.

The invention is based on the recognition that, although transillumination from one side of the turbid medium to the opposite side is not always possible, scattering of the light signal and relatively short range light paths on one side of the turbid medium close to the brim can provide relevant imaging data. This recognition is particularly true if natural fluorescence or a fluorescent agent is used in imaging an interior of the turbid medium. The fluorescence acts as a secondary light source enhancing the resolution and traceability of the location where the fluorescence originated. In this way the absence of information that would have been available in a situation of transillumination is compensated in that a kind of virtual transillumination is created, with a light path from the fluorescent material to the photodetector unit.

An embodiment of the device according to the invention is characterized in that at least a part of a surface of the brim facing the receiving volume has a substantially convex shape relative to the receiving volume. This embodiment has the advantage that the shape of the brim provides improved imaging conditions by enabling the optical coupling of the light source to the turbid medium and the turbid meaning to the photodetector unit.

In medical diagnostics, for instance, where the device may be used for imaging an interior of a female breast, a brim comprising a convex surface facing the turbid medium enables the imaging of the part of a breast close to a patient's armpit. The convex surface may be smooth or comprise one or more kinks.

A further embodiment of the device according to the invention is characterized in that the brim is optically coupled to at least two straight, crossing light guides. This embodiment has the advantage that light emanating from the receiving volume can be observed without the light reflecting off an inner wall of the light guide. The light guide could be an optical fiber, an endoscope, or simply a borehole in a solid material. The light guides are arranged such that their paths cross, but do not intersect each other.

A further embodiment of the device according to the invention is characterized in that the brim is optically coupled to a curved light guide. This embodiment has the advantage that curved light guides allows for light guides to cross each other easily and makes assembly of the light guides possible without introducing tight bends in the light guides. Tight bends will lead to optical losses. Tight bends may also lead to breaking of light guides, for instance, if these light guides are flexible optical fibers.

A further embodiment of the device according to the invention is characterized in that the brim is optically coupled to a flexible light guide. This embodiment has the advantage that a flexible light guide allows for easy assembly of light guides that cross each other without introducing tight bends in the light guides.

A further embodiment of the device according to the invention is characterized in that the device comprises a first receptacle and a second receptacle with the first receptacle being arranged for receiving the second receptacle, with the second receptacle bounding the receiving volume and comprising the brim and with the second receptacle being optically coupled to both the receiving volume and the first receptacle. This embodiment has the advantage that a second receptacle comprising a brim having one shape can be easily exchanged for a further second receptacle comprising a further brim having a different shape. If the wall of the second receptacle comprises a hollow volume, curved light guides may be used inside the hollow volume for optically coupling the second receptacle to the receiving volume and the first receptacle. A second receptacle comprising such a hollow volume comprising light guides has the advantage that it is light, allows for arbitrary crossing paths for light guides and has good manufacturability, for instance if the second receptacle comprises a first part and a second part that are coupled such that together they enclose the hollow volume.

A further embodiment of the device according to the invention is characterized in that the brim is a disk. In medical diagnostics, where the device may, for instance, be used for imaging an interior of a female breast, this embodiment is useful in case of a measurement geometry in which a breast is hanging freely through an opening in a structure supporting the patient. If the brim of the opening is defined by a disk arranged for optically coupling the light source to the turbid medium and the turbid medium to the photodetector unit, measurements near the patient's armpit become possible. The disk may be removable to allow the use of different disks having different sizes for the opening.

The object of the invention is further realized with a second receptacle, arranged to be inserted into a first receptacle, with the first receptacle being comprised in a device for imaging an interior of a turbid medium, said device comprising a light source for irradiating the turbid medium and a photodetector unit for detecting light emanating from the turbid medium as a result of irradiating the turbid medium and with the second receptacle bounding a receiving volume for receiving at least a part of the turbid medium, said receiving volume having an opening bound by a brim with the brim being arranged for optically coupling the light source to the turbid medium and the turbid medium to the photodetector unit. Use of a second receptacle has the advantage that a second receptacle comprising a brim having one shape can be easily exchanged for a further second receptacle comprising a further brim having a different shape. If the wall of the second receptacle comprises a hollow volume, curved light guides may be used inside the hollow volume for optically coupling the second receptacle to the receiving volume and the first receptacle. A second receptacle comprising such a hollow volume comprising light guides has the advantage that it is light, allows for arbitrary crossing paths for light guides and has good manufacturability, for instance if the second receptacle comprises a first part and a second part that are coupled such that together they enclose the hollow volume.

The medical image acquisition device according to the invention is defined in claim 9. According to the invention the medical image acquisition device is characterized in that at least a part of the brim is arranged for optically coupling the light source to the turbid medium and the turbid medium to the photodetector unit. If, for instance, the device is used to image an interior of a female breast, as is done in medical diagnostics, the device would benefit from any of the previous embodiments.

### BRIEF DESCRIPTION OF THE DRAWINGS

These and other aspects of the invention will be further elucidated and described with reference to the drawings, in which:

FIG. 1 schematically shows a device for imaging an interior of a turbid medium as known from prior art;

FIG. 2 schematically shows a cross-sectional drawing of a patient with one breast suspended in a receptacle having a convex shaped brim facing the breast;

FIG. 3 schematically shows a cross-sectional view of an embodiment of a receptacle comprising a removable second receptacle with the second receptacle comprising a convex shaped brim and curved light guides;

FIG. 4a schematically shows a receiving volume comprising an opening bound by a disk;

FIG. 4b schematically shows a cross-sectional side view of a receiving volume comprising an opening bound by a disk as shown in FIG. 4a;

FIG. 4c schematically shows a segment of a disk for bounding an opening with the disk comprising a grid of lamellas;

FIG. 5 schematically shows an embodiment of other medical image acquisition device according to the invention.

### DETAILED DESCRIPTION OF EMBODIMENTS

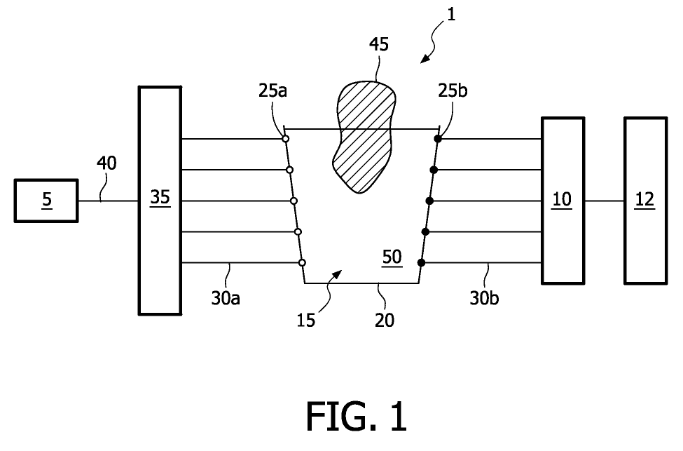

FIG. 1 schematically shows a device 1 for imaging an interior of a turbid medium as a known from prior art. The device 1 comprises a light source 5, a photodetector unit 10, a receiving volume 15 bound by a receptacle 20, said receptacle comprising a plurality of entrance positions for light 25a and exit positions for light 25b, and light guides 30a and 30b coupled to said entrance and exit positions. The device 1 further includes a selection unit 35 for coupling the input light guide 40 to a number of entrance positions for light selected from the plurality of entrance positions for light 25a in the receptacle 20. For the sake of clarity, entrance positions for light 25a and exit positions for light 25b have been positioned at opposite sides of the receptacle 20. In reality, however, both types of position may be distributed around the surface of the receptacle 20 facing the receiving volume 15. The device 1 further comprises an image reconstruction unit 12 for reconstructing an image of an interior of the turbid medium 45 based on the light detected by the photodetector unit 10. A turbid medium 45 is placed inside the receiving volume 15. The turbid medium 45 is then irradiated with light from the light source 5 from a plurality of positions by coupling the light source 5 using the selection unit 35 to successively selected entrance positions for light 25a. The light is chosen such that it is capable of propagating through the turbid medium 45. Light emanating from the receiving volume 15 as a result of irradiating the turbid medium 45 is detected from a plurality of exit positions using exit positions for light 25b and using photodetector 10. The detected light is then used to derive an image of an interior of the turbid medium 45. Deriving an image of an interior of the turbid medium 45 based on the detected light is possible as at least part of this light has traveled through the turbid medium 45 and, as a consequence, contains information relating to an interior of the turbid medium 45. The light has been intentionally chosen such that it is capable of propagating through the turbid medium 45. If, as may be the case in medical diagnostics, the device 1 is used for imaging an interior of a female breast, suitable light is, for instance, laser light within a wavelength range of 650 nm to 900 nm. In the receiving volume 15 the turbid medium 45 may at least partially be surrounded by a further medium 50 that may be used to counteract boundary effects stemming from the optical coupling of the turbid medium 45 with its surroundings. The optical characteristics of the further medium 50 at least partially surrounding the turbid medium 45 inside the receiving volume 15 must be such that characteristics, such as, for instance, the absorption coefficient match those of the turbid medium 45 being imaged for the wavelengths of light used for imaging an interior of the turbid medium 45. By matching optical characteristics boundary effects are significantly reduced. In FIG. 1 the receiving volume 15 is bound by a receptacle 20. However, this need not always be the case. Another embodiment of a device for imaging an interior of a turbid medium is that of a handheld device that may, for instance, be pressed against a side of a turbid medium. In that case, the receiving volume is the volume occupied by the part of the turbid medium from which light is detected as a result of irradiating the turbid medium.

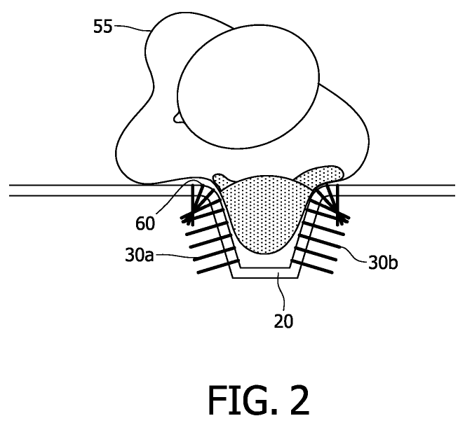

FIG. 2 schematically shows a cross-sectional drawing of a patient 55 with one breast suspended in a receptacle 20 having a convex shaped brim 60 facing the breast. The convex shaped brim 60 enables imaging an interior of the patient 55 near the patient's armpit. When looking for tumors in breast tissue, imaging of this area is very important as this is an area where there are lymph nodes. Moreover, breast cancer is often found on the lateral side of the breast relatively close to the armpit. Optically coupled to receptacle 20 are light guides 30a and 30b. Light guides 30a and 30b are arranged as straight light guides with different light guides 30a and 30b lying in different planes parallel to the plane of FIG. 2 so that different light guides 30a and 30b lying in different planes cross each other but do not intersect. Light guides 30a and 30b may be optical fibers, endoscopes, or simply boreholes in a solid material. Straight light guides have the advantage that light emanating from the receptacle 20 can be collected without the light reflecting off inner walls of the light guides. Having light reflect off inner walls of the light guides leads to optical losses. FIG. 2 clearly illustrates that without light guides 30a and 30b coupled to the convex shaped brim 60 a region of the patient's breast cannot be imaged with the possibility that possible tumors go undetected.

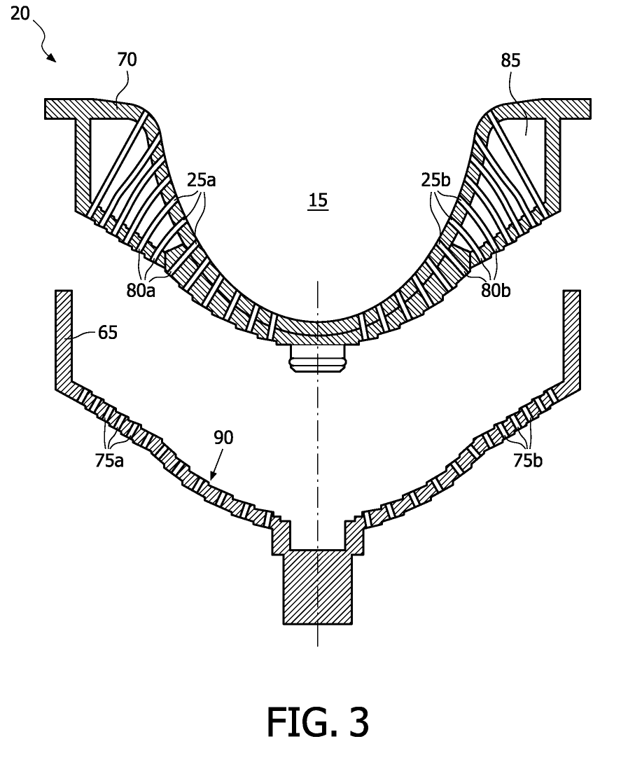

FIG. 3 schematically shows a cross-sectional view of a receptacle 20 comprising a first receptacle 65 and a removable second receptacle 70 with the removable second receptacle 70 having a convex shaped brim. The first receptacle 65 comprises optical coupling positions 75a and 75b for optically coupling the first receptacle 65 to the light source 5 and the photodetector unit 10, respectively. The second receptacle 70 comprises entrance positions for light 25a and exit positions for light 25b (see FIG. 1). Entrance positions for light 25a are optically coupled to optical coupling positions 75a using light guides 80a. Exit positions for light 25b are optically coupled to optical coupling positions 75b using light guides 80b. At least some of the light guides 80a and 80b may be curved to allow light guides to cross each other. An optical fiber may be used as a curved light guide. At least some of the light guides 80a and 80b may be flexible thus making the crossing of light guides easier than is the case with curved, but inflexible light guides. For the sake of clarity, entrance positions for light 25a have been depicted opposite of exit positions for light 25b. In reality, however, both types of position may be distributed over the surface of the second receptacle 70 the faces are receiving volume 15. The second receptacle 70 is positioned in the first receptacle 65 using steps 90. The assembly of the receptacle 20, the second receptacle 70, and the light guides 80a and 80b allows for the light guides to cross one another and makes assembly of the light guides possible without introducing tight bends. Tight bends will lead to optical losses and may also lead to the breaking of light guides. The second receptacle 70 need not be solid. In FIG. 3, the second receptacle 70 comprises a space 85. Depending on the size of the receiving volume 15 the space 85 may be bigger or smaller. In FIG. 3 the size of the receiving volume 15 is almost at a maximum as is clear from the fact that the surface of the second receptacle 70 that faces the receiving volume 15 and the surface of the second receptacle 70 that faces the first receptacle 65 are close to each other near the axis of symmetry of the second receptacle 70. If the size of the receiving volume 15 is smaller than depicted in FIG. 3, the two surfaces will be further apart allowing more room for the space 85. The assembly shown in FIG. 3 has the advantage that it is light as compared to a solid receptacle, enables arbitrary crossings of light guides 80a and 80b, and has good manufacturability. Clearly, curved light guides may also be coupled to the first receptacle 65 as an alternative to the situation shown in FIG. 2. If there is no need to collect light emanating from the receiving volume 15 without the light reflecting off inner walls of the light guides 30b (see FIG. 2), curved light guides have the advantage that they enable arbitrary crossings and simplify the optical coupling of the receiving volume 15 to the light source 5 and the photodetector unit 10.

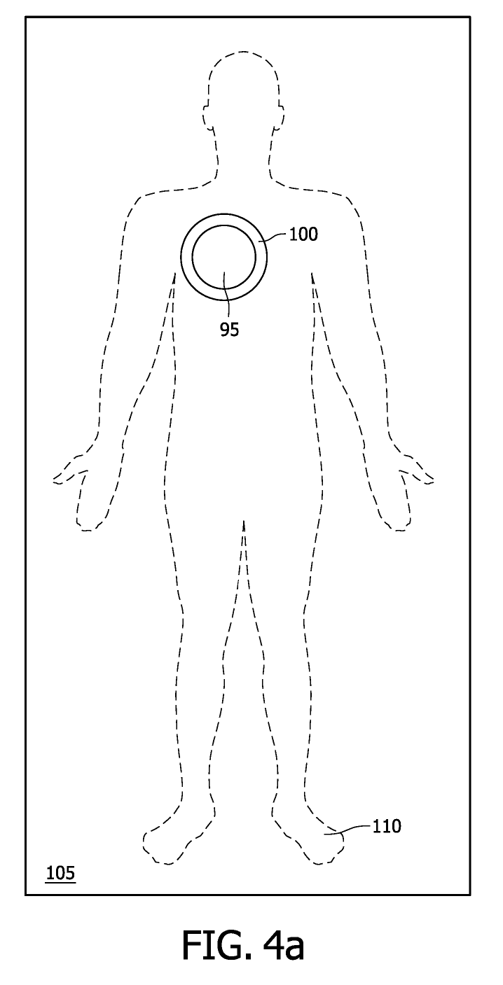

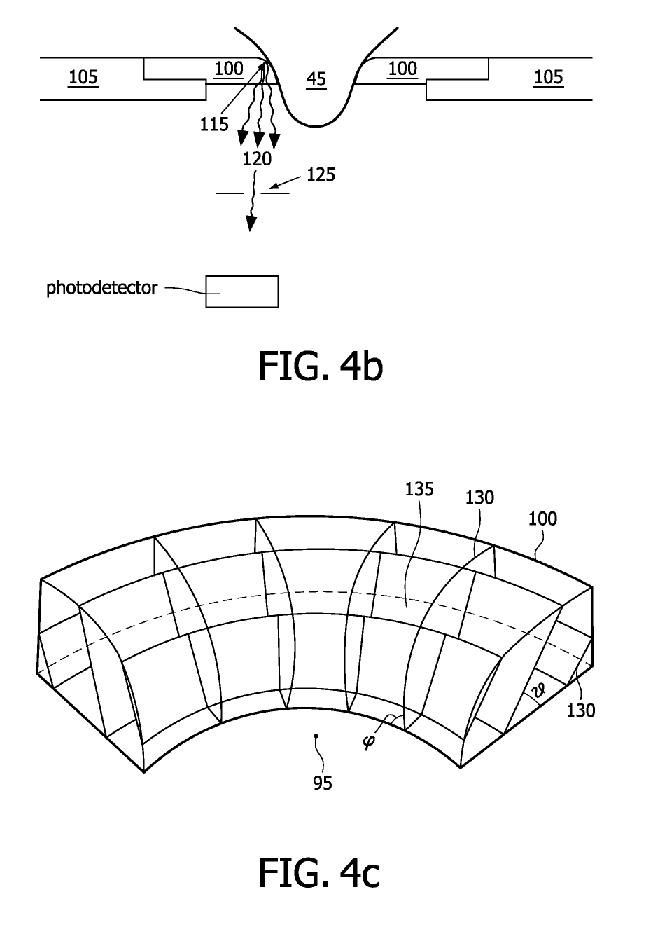

FIG. 4a schematically shows a top view of a receiving volume comprising an opening 95 bound by a disk 100. The disk 100 is arranged for optically coupling a light source (not shown in FIG. 4a) to the turbid medium 45 and the turbid medium 45 to a photodetector unit 10 (not shown in FIG. 4a). This can, for instance, be achieved by making the disk 100 of a material that is transparent to the light emanating from the light source and the receiving volume 15 such as glass or certain plastics. The disk 100 may be removable to allow the use of different disks having different sizes for the opening 95. The use of different disks having different sizes for the opening 95 enables the proper positioning of turbid mediums 45 of different sizes in the opening 95. The setup shown in FIG. 4a is advantageous in, for instance, medical devices for imaging tumors in breast tissue in which a measurement geometry is used of a hole through which a breast is hanging freely. The setup shown in FIG. 4a comprises a support structure 105 looking much like a bed for supporting a patient 110 who is indicated by the dashed line. In FIG. 4a the patient 110 is lying face down on the bed-like support structure 105.

FIG. 4b schematically shows a cross-sectional side view of a receiving volume comprising an opening 95 bound by a disk 100 as shown in FIG. 4a. The opening 95 and disk 100 are comprised in a support structure 105 supporting the patient 110. A breast of the patient 110 is hanging freely through the opening 95 in the support structure 105. The patient 110 is supported on one side of the support structure 105, whereas a light ray 115 from a light source (not shown in FIG. 4b) impinges on the patient's breast while coming from the other side of the support structure 105. The disk 100 is arranged for optically coupling the light source to the turbid medium 45, in this case the patient's breast, and the turbid medium 45 to a photodetector unit. This means that light rays coming from one side of the disk 100 can pass through at least a part of the disk 100 to end up at the other side of the disk 100. In FIG. 4b this was already illustrated by light ray 115. Light rays 120 generated as a result of irradiating the breast with light ray 115 are coupled to the photodetector unit, for instance by channeling the light rays 120 through a hole 125 in order to restrict the direction of light before been detected by the photodetector unit. Alternatively, the disk 100 may comprise a grid of lamellas that are themselves opaque to the light emanating from the light source and the turbid medium 45 (see FIG. 4c). If irradiation of the turbid medium 45, in this case the patient's breast, results in the emission from the turbid medium 45 of fluorescence light resulting from natural fluorescence or from a fluorescent agent present in the turbid medium 45, the fluorescence acts as a secondary light source enhancing the resolution and traceability of the location where the fluorescence originated.

FIG. 4c schematically shows a segment of a disk 100 for bounding an opening 95 with the disk 100 comprising a grid of lamellas 130. The grid of lamellas 130 is arranged such that the grid 130 forms a plurality of optical channels 135 with each optical channel 135 being able to conduct light to and from a specific area of the surface of the turbid medium 45 (not shown in FIG. 4c). The grid of lamellas 130 ensures that light emanating from the receiving volume 15 and next from a specific optical channel 135 formed by the grid of lamellas 130 emanates from a specific area of the surface of the turbid medium 45. Hence, this light carries information relating to a specific part of the turbid medium 45 only and is not mixed with light that emanated from other areas of the surface of the turbid medium 45 and that carries information relating to other parts of the turbid medium 45. As discussed in relation to FIG. 4b, the grid of lamellas 130 is opaque to the light emanating from the light source and the receiving volume 15. The grid of lamellas 130 may be positioned in the disk 100 at angles θ and φ with the values of θ and φ depending on, for instance, the position from which light from the light source is coupled into the disk 100 and on how the means for detecting light emanating from the receiving volume 15 and passing through the disk 100 are arranged.

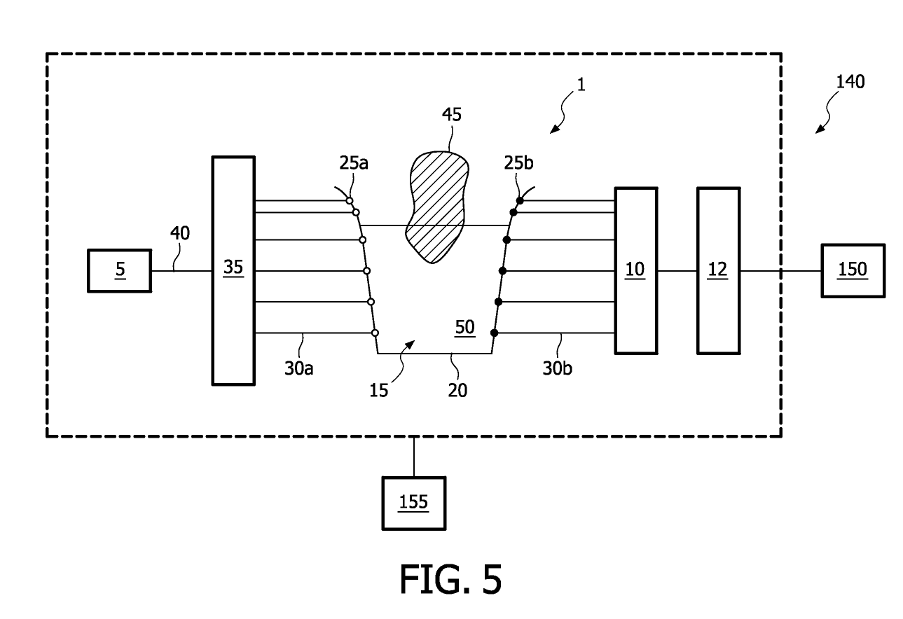

FIG. 5 schematically shows an embodiment of a medical image acquisition device 140 according to the invention. Shown inside the dashed square is essentially the device 1 shown in FIG. 1. However, the receiving volume 15 now comprises an opening bound by a brim according to the invention as shown in FIG. 2. The medical image acquisition device 140 further comprises a screen 150 for displaying a reconstructed image and an input interface 155, for instance, a keyboard enabling an operator to interact with the medical image acquisition device 140.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. The word “comprising” does not exclude the presence of elements or steps other than those listed in a claim. The word “a” or “an” preceding an element does not exclude the presence of a plurality of such elements. In the system claims enumerating several means, several of these means can be embodied by one and the same item of computer readable software or hardware. The mere fact that certain measures are recited in mutually different dependent claims does not indicate that a combination of these measures cannot be used to advantage.

1. A device (1) for imaging an interior of a turbid medium (45) comprising:
a) a receiving volume (15) for receiving at least a part of the turbid medium (45), said receiving volume (15) comprising an opening bound by a brim;
b) a light source (5) for irradiating the turbid medium (45);
c) a photodetector unit (10) for detecting light emanating from the receiving volume (15) as a result of irradiating the turbid medium (45)
characterized in that
at least a part of the brim is arranged for optically coupling the light source (5) to the turbid medium (45) and the turbid medium (45) to the photodetector unit (10).

2. A device as claimed in claim 1, wherein at least a part of a surface of the brim facing the receiving volume (15) has a substantially convex shape relative to the receiving volume (15).

3. A device as claimed in claim 1, wherein the brim is optically coupled to at least two straight, crossing light guides (30a, 30b).

4. A device as claimed in claim 1, wherein the brim is optically coupled to a curved light guide (30a, 30b).

5. A device as claimed in claim 1, wherein the brim is optically coupled to a flexible light guide (30a, 30b).

6. A device as claimed in claim 1 wherein the device comprises a first receptacle (65) and a second receptacle (70) with the first receptacle (65) being arranged for receiving the second receptacle (70), with the second receptacle (70) bounding the receiving volume (15) and comprising the brim and with the second receptacle (70) being optically coupled to both the receiving volume (15) and the first receptacle (65).

7. A device as claimed in claim 1, wherein the brim is a disk (100).

8. A second receptacle (70), arranged to be inserted into a first receptacle (65) with the first receptacle (65) being comprised in a device for imaging an interior of a turbid medium (45), said device comprising a light source (5) for irradiating the turbid medium (45) and a photodetector unit (10) for detecting light emanating from the turbid medium (45) as a result of irradiating the turbid medium (45) and with the second receptacle (70) bounding a receiving volume (15) for receiving at least a part of the turbid medium (45), said receiving volume (15) having an opening bound by a brim with the brim being arranged for optically coupling the light source (5) to the turbid medium (45) and the turbid medium (45) to the photodetector unit (10).

9. A medical image acquisition device (140) comprising:
a) a receiving volume (15) for receiving at least a part of a turbid medium (45), said receiving volume (15) comprising an opening bound by a brim;
b) a light source (5) for irradiating the turbid medium (45);
c) a photodetector unit (10) for detecting light emanating from the receiving volume (15) as a result of irradiating the turbid medium (45)
wherein
at least a part of the brim is arranged for optically coupling the light source (5) to the turbid medium (45) and the turbid medium (45) to the photodetector unit (10).
